(19) United States
(12) Patent Application Publication
(10) Pub. No.: US 2019/0333649 A1
(43) Pub. Date: Oct. 31, 2019

(54) DOCTOR-PATIENT VIDEO CHAT STORYBOARDING

(71) Applicant: Covidien LP, Mansfield, MA (US)

(72) Inventors: Jonathan James Woodward, Annapolis, MD (US); Amit Mukherjee, Elkridge, MD (US); Mark Kamensek, Annapolis, MD (US)

(21) Appl. No.: 15/962,251

(22) Filed: Apr. 25, 2018

(51) Int. Cl. G16H 80/00; H04N 7/15; G16H 10/60; G16H 30/20; G16H 15/00

(52) U.S. Cl. CPC G16H 80/00; H04N 7/15; G16H 15/00; G16H 30/20; G16H 10/60

(57) ABSTRACT

Methods, systems, and devices for video conferencing between a patient and a clinician are described. The method may include receiving a video image of a video conference between the patient and the clinician. The method may also receive an indication of medical information associated with the patient to be displayed during the video conference. The method may further include configuring a viewing window of a video conference device to display the video image and the medical information during the video conference. The configuration of the viewing window may be based on a characteristic of the medical information to be displayed.

### BACKGROUND

The following relates generally to video conferencing between a patient and a clinician, and more specifically to doctor-patient video chat storyboarding.

In a healthcare facility such as a hospital, physiological parameters of the patient (e.g., heart rate, respiratory rate, blood pressure) may be monitored by one or more medical devices. The medical devices may be battery powered and may wirelessly transmit measured patient data over a wireless network within the hospital, thereby allowing the patient to move freely through the hospital while being monitored. Clinicians may remotely monitor the patient by accessing the patient data at a central nurse station or on any web enabled device connected to the network (e.g., smartphone or tablet).

The medical devices may be configured to transmit patient data over a network outside of the hospital, thereby allowing the patient to return home and continue patient care outside the hospital. In addition, the patient may conduct a video conference with a clinician as part of their at-home care plan. For example, the patient may set up a video conference between the patient and the clinician to answer questions or to perform follow-up diagnosis or consultations.

### SUMMARY

The described features generally relate to methods, systems, devices, or apparatuses that support doctor-patient video chat storyboarding. A video conference server may receive a live video feed between a patient and clinician participating in a video conference. The video feed may come from web camera-enabled devices, such as phones, tablets, computers, or the like. The video conference server may also receive medical information to be displayed on a viewing window of one or both of the devices at the same time of the live video feed. Therefore, the video conference server may configure the viewing window of one or both of the devices to display the video feed and the medical information, thereby enabling the clinician to augment the video consultation with relevant medical data in real time. The ability to storyboard the medical information to the patient in this way may increase the effectiveness of the information being conveyed by the clinician, which may improve patient compliance and ultimately patient outcomes in the at-home care setting.

In some examples, the video feed and the medical information may be arranged in the viewing window according to characteristics of the medical information. For example, the medical information may be arranged with respect to the video feed according the size, orientation, or other characteristics of the medical information. The resolution and size of the video feed may also be modified based on the medical information to be displayed. In some cases, the clinician or patient may add annotations to the medical information or video feed.

A method for video conferencing between a patient and a clinician is described. The method may include receiving a video image of a video conference between the patient and the clinician, receiving an indication of medical information associated with the patient to be displayed during the video conference, and configuring a viewing window to display the video image and the medical information during the video conference. In some examples of the method described herein, the characteristic of the medical information may comprise an orientation of the medical information, a size of the medical information relative to a size of the video image, whether the medical information is dynamically or statically updated, or a combination thereof.

Some examples of the method described herein may further include arranging the video image in a first portion of the viewing window and arranging the medical information in a second portion of the viewing window, wherein the location of the video image in the first portion is different than the location of the medical information in the second portion. Some examples of the method described herein may further include arranging the video image and the medical information such that the medical information at least partially overlaps the video image.

In some examples, configuring the viewing window may comprise modifying a size of the video image based at least in part on a size of the medical information relative to the size of the video image, an orientation of the medical information, or both. In other examples, configuring the viewing window may comprise adjusting a resolution of the video image based at least in part on a resolution of the medical information. Some examples of the method described herein may further include receiving a request for the medical information from at least one of the clinician, the patient, or both, and wherein the medical information to be displayed is based at least in part on the received request. Some examples of the method described herein may further include receiving an indication of an annotation to the medical information from the clinician, the patient, or both and arranging the annotation to be displayed with the medical information in the viewing window.

Some examples of the method described herein may further include operations, features, means, or instructions for retrieving the medical information from a database, a sensor associated with the patient, an input by the patient or the clinician during the video conference, or a combination thereof. Some examples of the method described herein may further include operations, features, means, or instructions for transmitting an indication of the medical information that was displayed during the video conference to a server for storage. Some examples of the method described herein may further include displaying the video image and the medical information in the viewing window according to the configuration. In some examples of the method described herein, the medical information may comprise medical data associated with the patient. In some examples of the method described herein, the medical information may be represented as a chart, a graph, a report, an image, a live data stream, or a combination thereof.

### BRIEF DESCRIPTION OF THE DRAWINGS

FIG. 1 illustrates an example of a wireless patient monitoring system that supports doctor-patient video chat storyboarding in accordance with aspects of the present disclosure.

FIG. 2 illustrates an example of video conferencing between a patient and a clinician that supports doctor-patient video chat storyboarding in accordance with aspects of the present disclosure.

FIG. 3A illustrates examples of an oriented video conference configuration that supports doctor-patient video chat storyboarding in accordance with aspects of the present disclosure.

FIG. 3B illustrates examples of a sized video conference configuration that supports doctor-patient video chat storyboarding in accordance with aspects of the present disclosure.

FIG. 3C illustrates examples of a dynamically updated video conference configuration that supports doctor-patient video chat storyboarding in accordance with aspects of the present disclosure.

FIG. 3D illustrates examples of an annotated video conference configuration that supports doctor-patient video chat storyboarding in accordance with aspects of the present disclosure.

FIG. 4 illustrates an example process flow that supports doctor-patient video chat storyboarding in accordance with aspects of the present disclosure.

FIGS. 5 through 7 show block diagrams of a device that supports doctor-patient video chat storyboarding in accordance with aspects of the present disclosure.

FIG. 8 illustrates a block diagram of a system including a video conference server that supports doctor-patient video chat storyboarding in accordance with aspects of the present disclosure.

FIGS. 9 through 12 illustrate methods for doctor-patient video chat storyboarding in accordance with aspects of the present disclosure.

### DETAILED DESCRIPTION

In some home-based monitoring systems, a patient may return home from the healthcare facility with instructions for at-home patient care. If the patient returns home with questions regarding at-home treatment or concerns regarding a medical condition, the patient may initiate a video conference with the clinician. For example, a video communication system that facilitates the video conference may receive a video image of the patient or clinician from video-enabled devices such as a phone or tablet. The video communication system may also receive an indication of medical information associated with the patient that the clinician wishes to display to the patient during the video conference.

The video communication system may then configure the viewing window of the patient's device to display the video image and the medical information at the same time. For example, the video feed (e.g., including the video image) between the patient and the clinician may display the medical information to tell a story (e.g., show trends, cause and effect relationships, etc.) and convey the information to the patient in a storyboard format. In some cases, configuring the viewing window to display the information in a storyboard format may allow the patient to understand the information more clearly and increase patient compliance with the clinician's prescribed treatment plan.

In some examples, the medical information and the video image may be arranged in a specific configuration according to the characteristics of the medical information. That is, the medical information may be arranged in a viewing window according to the size, shape, orientation, and dynamic nature of the medical information. For example, the medical information may be arranged in a vertical direction if the medical information contains a vertically-oriented graphic. In some cases, the medical information may be arranged side by side (e.g., non-overlapping) with respect to the video image. In other examples, the medical information may be arranged in an overlapping configuration with the video image.

Based on the availability of wireless communications, the resolution of the video image and the resolution of the medical information may be adjusted. In some cases, a video communication system may modify the size of the video image or the medical information with respect to each other. The medical information may include medical data associated with patient. For example, the medical data may be conveyed though a chart, graph, report, image, live data stream, or a combination thereof.

During the video conference, the clinician may add notes (e.g., annotate) the video image or medical information to convey a message to the patient. For example, the clinician may circle a point of interest in the video image to discuss in further detail with the patient. In some examples, the clinician may annotate a graph to show a cause and effect relationship if the patient misses a medication dose.

In some cases, the viewing window of the video conference may be configured to avoid increased risk to the patient and prevent future medical complications. That is, the configuration of the video conference enables the patient and clinician to have a constructive discussion by showing the medical information of interest and the video feed at the same time.

Aspects of the disclosure are initially described in the context of a wireless patient monitoring system. Aspects of the disclosure are further illustrated by and described with reference to apparatus diagrams, system diagrams, and flowcharts that relate to doctor-patient video chat storyboarding.

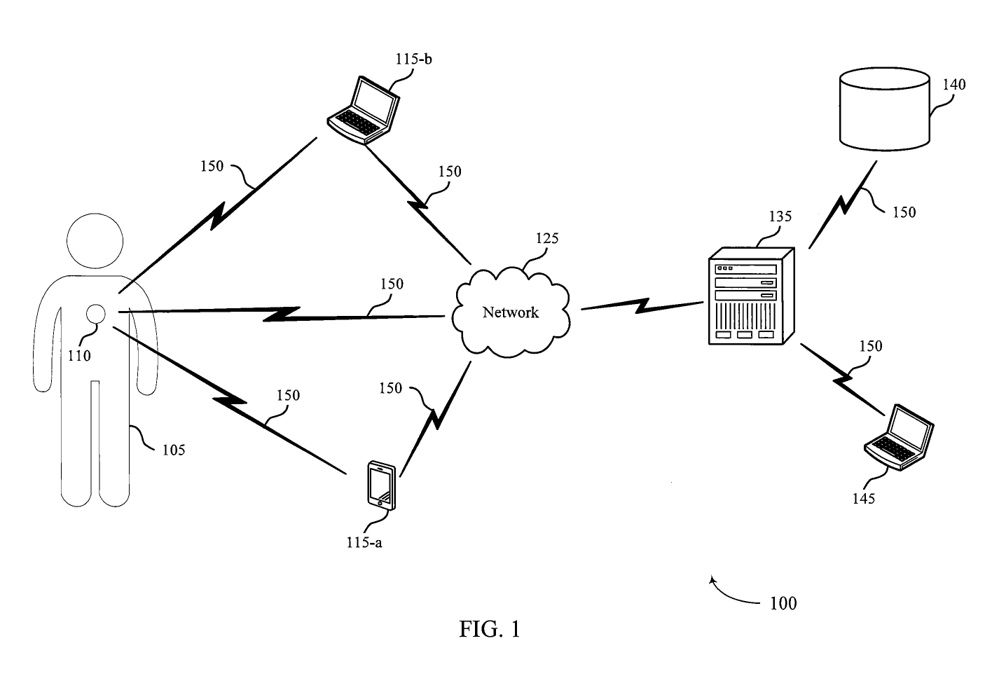

FIG. 1 illustrates an example of a wireless patient monitoring system 100 that supports doctor-patient video chat storyboarding in accordance with various embodiments of the present disclosure. The wireless patient monitoring system 100 may include a patient 105 wearing, carrying, or otherwise coupled with a medical device 110. Although a single medical device 110 is shown, multiple medical devices 110 may be coupled to the patient 105. The patient 105 may be a patient in a hospital, nursing home, home care, a medical facility, or another care facility. The medical device 110 may transmit signals via wireless communications links 150 to computing devices 115 or to a network 125.

The medical device 110 may include one or more sensors configured to collect a variety of physiological parameters as well as information related to the location and movement of the patient 105. For example, the medical device 110 may include a pulse oximetry (SpO2) sensor, a capnography sensor, a heart rate sensor, a blood pressure sensor, an electrocardiogram (ECG) sensor, a respiratory rate sensor, a glucose level sensor, a depth of consciousness sensor, a body temperature sensor, an accelerometer, a global positioning sensor, a sensor which triangulates position from multiple local computing devices 115, or any other sensor configured to collect physiological, location, or motion data associated with the patient 105.

The medical device 110 may be coupled with the patient 105 in a variety of ways depending on the data being collected. For example, the medical device 110 may be directly coupled with the patient 105 (e.g., physically connected to the patient's chest, worn around the patient's wrist, attached to the patient's finger, or positioned over the patients nose or mouth). The data collected by the medical device 110 may be wirelessly transmitted to either the computing devices 115 or to the remote computing device 145 (via the network 125 and central station 135). Data transmission may occur via, for example, frequencies appropriate for a personal area network (such as Bluetooth, Bluetooth Low Energy (BLE), or IR communications) or local (e.g., wireless local area network (WLAN)) or wide area network (WAN) frequencies such as radio frequencies specified by IEEE standards (e.g., IEEE 802.15.4 standard, IEEE 802.11 standard (Wi-Fi), IEEE 802.16 standard (WiMAX), etc.).

Computing device 115-a may be a wireless device such as a tablet, cellular phone, personal digital assistant (PDA), a dedicated receiver, or other similar device or a spatially distributed network of devices configured to receive signals from the medical device 110. Computing device 115-b may be a wireless laptop computer, a clinician Workstation on Wheels, or a smart hospital bed configured to receive signals from the medical device 110. Computing device 115-a may also be configured to receive a video image and medical information and configure the video image and medical information in a viewing window.

The computing devices 115 may be in communication with a central station 135 via network 125.

The medical device 110 may also communicate directly with the central station 135 via the network 125. The central station 135 may be a server or a central nurse station located within the hospital or in a remote location. The central station 135 may be in further communication with one or more remote computing devices 145, thereby allowing a clinician to remotely monitor the patient 105. The central station 135 may also be in communication with various remote databases 140 where the collected patient data may be stored. In some cases, the remote databases 140 include electronic medical records (EMR) applications for storing and sharing patient data.

In accordance with various embodiments, methods and apparatuses are described for video Conferencing between a patient and clinician. When patient 105 returns home after an appointment with the clinician, patient 105 may be monitored based on a medical condition or patient 105 may have follow-up questions for the clinician based on their medical condition. In that case, a video conference server (e.g., central station 135) may receive a video image from patient 105 or clinician to participate in a video conference. The video conference server may also receive medical information associated with patient 105 to be displayed during the video conference, and may retrieve the medical information from database 140. For example, video conference server may configure the viewing window of a computing device 115 to display the video image and medical information in a format that allows the user to view the video image and medical information at the same time.

In some examples, the video conference server may configure the viewing window to display the video image and medical information according to the size, shape, orientation, and dynamic characteristics associated with the medical information. For example, the video image may be arranged as to partially overlap the medical information. In some cases, the video image and the medical information may be arranged in a non-overlapping configuration. That is, the size of the video image may be modified based on the size or orientation of the medical information.

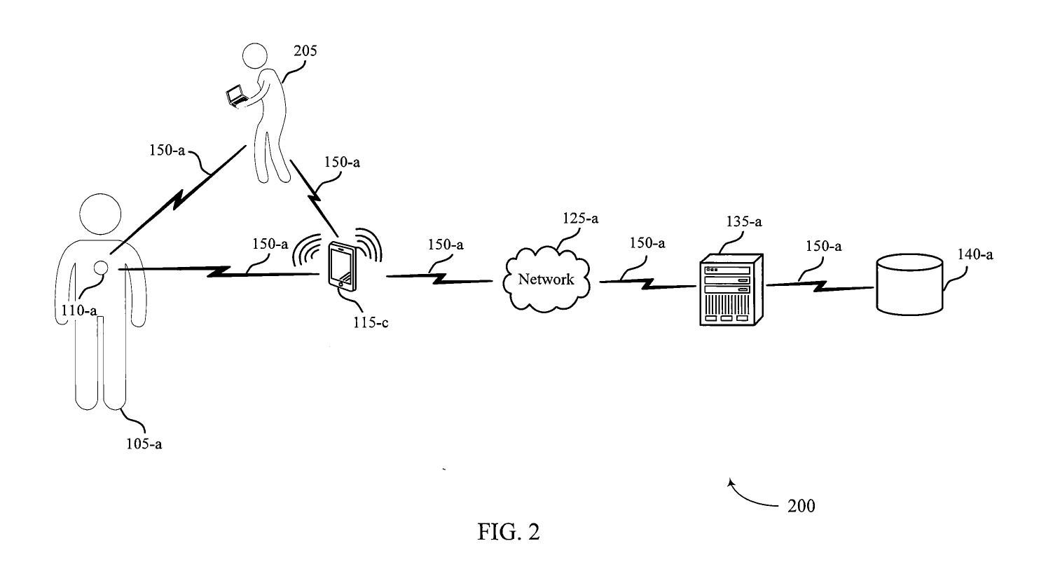

FIG. 2 illustrates an example of a system for video conferencing between a patient and a clinician system 200 that supports doctor-patient video chat storyboarding in accordance with various aspects of the present disclosure. Clinical system 200 may be an example of aspects of wireless patient monitoring system 100 and may include a patient 105-a wearing, carrying, or otherwise coupled with a medical device 110-a. Clinical system 200 may be in communication with clinician 205.

Patient 105-a may communicate bidirectionally via wired or wireless communication links 150-a to video conference device 115-c. Video conference device 115-c may be an example of aspects of computing device 115. Video conference device 115-c may also be an example of a device that receives a video feed (e.g., video image) and medical information to be displayed during a video conference between patient 105-a and clinician 205. In some cases, video conference device 115-c may be a tablet, cellular phone, or a web camera configured to initiate a video conference between patient 105-a and clinician 205. In some examples, the video feed and medical information may be configured in a video screen (e.g., viewing window) of the video conference device 115-c to display both the video feed and the medical information.

Video conference device 115-c may communicate bidirectionally via wired or wireless communication links 150-a to video conference server 135-a via network 125-a (e.g., the Internet). Video conference server 135-a may also communicate bidirectionally via wired or wireless communication links 150-a to database 140-a to retrieve medical information. In some cases, medical information displayed during the video conference may be transmitted to a server (e.g., via network 125-a) and stored. Video conference server 135-a may be an example of aspects of central station 135.

In some cases, video conference server 135-a may receive a request for medical information from patient 105-a, clinician 205, or both. For example, patient 105-a may request to view lab results from a recent appointment. In other examples, clinician 205 may request to view a part of the body where patient 105-a experiences pain. Video conference server 135-a may also receive medical information associated with patient 105-a from medical device 110-a (e.g., sensor) coupled to patient 105-a. In some cases, video conference server 135-a may receive medical information to display based on an input by patient 105-a or clinician 205 during the video conference. Therefore, clinician 205 may also communicate bidirectionally via wired or wireless communication links 150-a to video conference device 115-c.

Video conference server 135-a may configure the video screen of the video conference device 115-c to display the video feed and the medical information. For example, video conference server 135-a may configure the video screen to display both the video feed and the medical information by arranging the video feed and the medical information with respect to each other. In some examples, video conference server 135-a may arrange the video feed and the medical information based on characteristics of the medical information (e.g., size, orientation, etc.). Video conference server 135-a may also arrange the video feed and medical information to overlap or align (e.g., not overlap). Based on the configuration, video conference server 135-a may display the medical information and the video image during a video conference between patient 105-a and clinician 205.

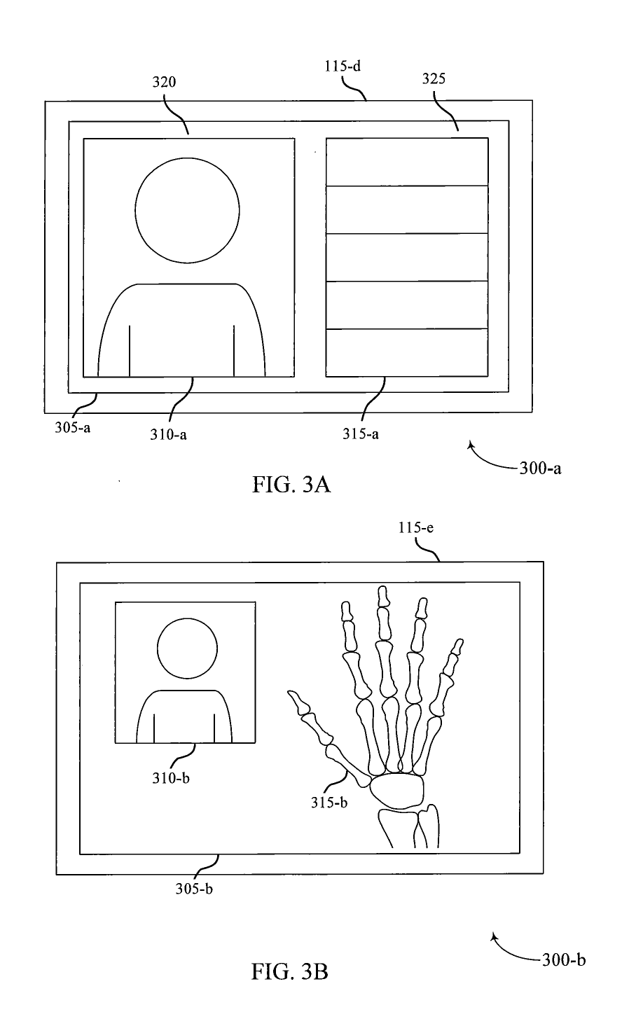

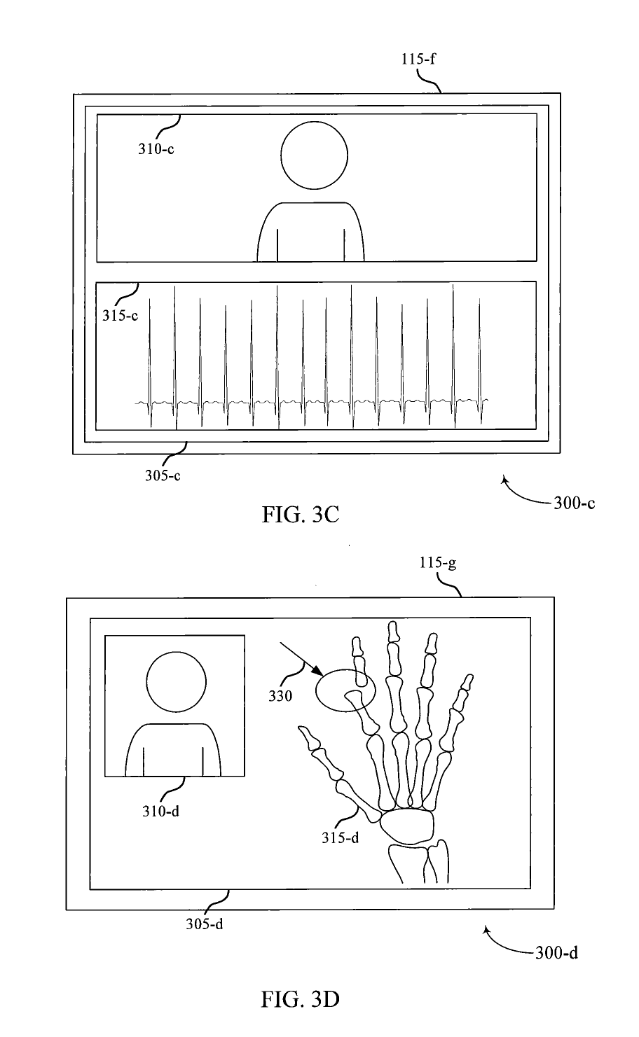

FIG. 3A illustrates an example of a sized video conference configuration 300-a that supports doctor-patient video chat storyboarding in accordance with various aspects of the present disclosure. Video conference configuration 300-a may be an example of aspects of clinical system 200 and may include video conference device 115-d, viewing screen 305-a (e.g., viewing window), video feed 310-a (video image), and medical information 315-a.

Video conference device 115-d may receive video feed 310-a and medical information 315-a during a video conference between a patient and a clinician, as described in reference to FIGS. 1-2. As described below, a video conference server may configure viewing screen 305-a to display video feed 310-a and medical information 315-a at the same time. For example, video feed 310-a may be arranged with respect to medical information 315-a. In some cases, viewing screen 305-a may be a single screen.

Based on the orientation of medical information 315-a, video feed 310-a may be arranged accordingly in viewing screen 305-a. For example, if medical information 315-a is orientated in a vertical direction, video feed 310-a may be arranged next to medical information 315-a in a vertical direction. In other examples, if medical information 315-a is oriented in a horizontal direction, video feed 310-a may be arranged above or below medical information 315-a in a horizontal direction. The orientation of the medical information 315-a (or any other characteristic of the medical information 315-a that affects how the information is viewed) may be determined from metadata associated with the medical information 315-a.

Video feed 310-a and medical information 315-a may also be arranged according to a location in viewing screen 305-a. In some examples, viewing screen 305-b may be configured with preset locations for medical information 315-b and video feed 310-b to be displayed. For example, video feed 310-a may be arranged in first portion 320 of viewing screen 305-a and medical information 315-a may be arranged in second portion 325 of viewing screen 305-a. In some cases, first portion 320 and second portion 325 may be different (e.g., video feed 310-a and medical information 315-a do not overlap). In other examples, first portion 320 and second portion 325 may the same (e.g., video feed 310-a and medical information 315-a may overlap or at least partially overlap).

In some cases, the size of video feed 310-a may be modified based on the orientation of medical information 315-a. For example, if medical information 315-a is oriented in a vertical direction, the size of video feed 310-a may be reduced in a horizontal direction and increased in a vertical direction to accommodate the orientation of medical information 315-a. In other examples, if medical information 315-a is oriented in a horizontal direction, the size of video feed 310-a may be reduced in a vertical direction and increased in a horizontal direction. That is, the arrangement of medical information 315-a with respect to video feed 310-a based on the orientation of medical information 315-a may modify the size of video feed 310-a.

In some examples, the resolution of video feed 310-a may be adjusted based on the resolution of medical information 315-a. For example, the resolution of video feed 310-a may decrease if the resolution of medical information 315-a requires a higher resolution than video feed 310-a. In other examples, the resolution of video feed 310-a may increase if the resolution of video feed 310-a requires a higher resolution than medical information 315-a. For example, an x-ray displayed in viewing screen 305-a may require a higher resolution than video feed 310-a to view the details associated with the x-ray. Similarly, medical information 315-a may be of greater interest to the patient or clinician during the video conference call; therefore, the resolution of medical information 315-a may be adjusted to highlight the point of interest and improve the efficiency of the video conference. In other examples, resolution of video feed 310-a or medical information 315-a may be adjusted based on an available bandwidth associated with the video conference.

Medical information 315-a may include medical data associated with the patient. For example, medical information may be represented as a chart, a graph, a report, an image, a live data stream, or combination thereof. In other examples, medical information may be represented as a chart, a text stream, or a combination thereof. For example, an image may include an image of the medical condition associated with the patient or an x-ray. A chart, graph, report, or table may illustrate the medical conditions associated with the patient in a storyboard format. That is, the chart, graph, report, or table may show a cause and effect relationship or data trend associated with the medical condition of the patient. In some cases, the live data stream may report data from the medical device coupled to the patient. In some examples, the text stream may include subtitles or patient care instructions from the clinician. That is, medical information 315-a may be retrieved from a database, a sensor associated with the patient, manual input by the patient or the clinician into the video conference system, or audio input by the patient or the clinician.

FIG. 3B illustrates an example of a sized video conference configuration 300-b that supports doctor-patient video chat storyboarding in accordance with various aspects of the present disclosure. Video conference configuration 300-b may be an example of aspects of clinical system 200 and may include video conference device 115-e, viewing screen 305-b (e.g., viewing window), video feed 310-b (video image), and medical information 315-b.

Video conference device 115-e may receive video feed 310-b and medical information 315-b during a video conference between a patient and a clinician, as described in reference to FIGS. 1-2. As described below, a video conference server may then configure viewing screen 305-b to accommodate video feed 310-b and medical information 315-b in a viewable format for the user. For example, video feed 310-b may be arranged with respect to medical information 315-b to display both video feed 310-b and medical information 315-b.

Medical information 315-b may be arranged in viewing screen 305-b according to the size of medical information 315-b relative to the size of video feed 310-b. For example, medical information 315-b may be sized larger than video feed 310-b to view details associated with medical information 315-b. Therefore, video feed 310-b may arranged in a smaller location in viewing screen 305-b. In some examples, medical information 315-b may include details to be viewed in a larger display format (e.g., an x-ray, an electrocardiogram (ECG) graph, etc.). Therefore, the size of video feed 310-b may be modified based on the size of medical information 315-b to display medical information 315-b to the user in a viewable and efficient format.

FIG. 3C illustrates an example of a dynamically updated video conference configuration 300-c that supports doctor-patient video chat storyboarding in accordance with various aspects of the present disclosure. Video conference configuration 300-c may be an example of aspects of clinical system 200 and may include video conference device 1154, viewing screen 305-c (e.g., viewing window), video feed 310-c (video image), and medical information 315-c.

Video conference device 115-f may receive video feed 310-c and medical information 315-c during a video conference between a patient and a clinician, as described in reference to FIGS. 1-2. As described below, a video conference server may then configure viewing screen 305-c to display video feed 310-c and medical information 315-c at the same time. For example, medical information 315-c may be arranged with respect to video feed 310-c based on medical information 315-c characteristics.

Based on the characteristics of medical information 315-c, video feed 310-c may be arranged accordingly in viewing screen 305-c. For example, medical information 315-c may be dynamically or statically updated. That is, the medical sensor coupled to the patient may stream a live feed of the results (e.g., vital signs) from the medical sensor (e.g., an ECG device) to the viewing screen 305-c. In that case, medical information 315-c may be dynamically updated. For example, medical information 315-c may be arranged in a horizontal direction to display the dynamically updated medical information 315-c, and video feed 310-a may also be displayed in a horizontal direction to accommodate medical information 315-c. Alternatively, medical information 315-c may be arranged with respect to video feed 310-c if medical information 315-c is statically updated.

FIG. 3D illustrates an example of an annotated video conference configuration 300-d that supports doctor-patient video chat storyboarding in accordance with various aspects of the present disclosure. Video conference configuration 300-d may be an example of aspects of clinical system 200 and may include video conference device 115-g, viewing screen 305-d (e.g., viewing window), video feed 310-d (video image), and medical information 315-d.

Video conference device 115-g may receive video feed 310-d and medical information 315-d during a video conference between a patient and a clinician, as described in reference to FIGS. 1-2. As described below, a video conference server may configure viewing screen 305-d to display video feed 310-d, medical information 315-b, and annotations to video feed 310-d or medical information 315-b.

In some cases, video conference device 115-g may receive an annotation from the patient or the clinician. The patient or clinician may add content to viewing screen 305-d and collect additional information related to the medical condition of the patient. For example, the clinician or the patient may add note 330 (e.g., annotation) to medical information 315-d. Note 330 may be an example of arrows, drawings, highlighted regions of medical information 315-d, or text. In some cases, note 330 may overlay medical information 315-d or may be adjacent to medical information 315-d. A video conference server may arrange note 330 to be displayed with medical information 315-d.

In other examples, the clinician or patient may add note 330 to video feed 310-d. For example, if the clinician discusses a medical condition displayed by the patient in video feed 310-d, the clinician may add note 330 to circle or highlight the medical condition. Additionally, clinician may add note 330 in the form of a text stream to display at-home patient instructions. The video conference server may also store video feed 310-d, medical information 315-d, and note 330 for the patient's medical history and also as a reference for future complications or questions.

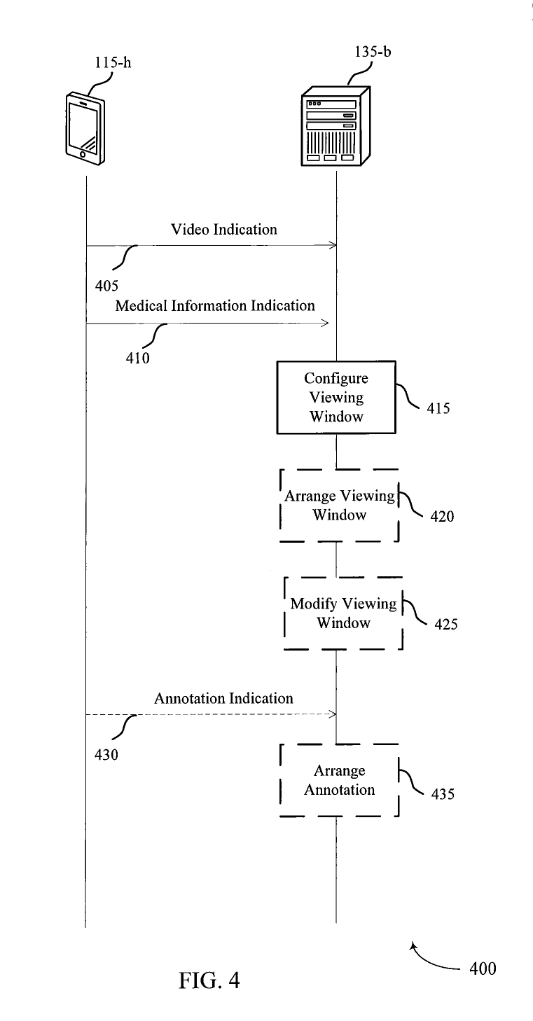

FIG. 4 illustrates an example of a process flow 400 that supports doctor-patient video chat storyboarding in accordance with various aspects of the present disclosure. Process flow 400 may include video conference device 115-h and video conference server 135-b, which may be respective examples of a computing device 115 and central station 135 as described with reference to FIGS. 1 and 2. The video conference device 115-h may represent a device being used by the patient, the clinician, or both. That is, depending on the particular feature being described below, the signaling being transmitted from the video conference device 115-h to the video conference server 135-b may be coming from the patient, the clinician, or both. Alternative examples of the following may be implemented, where some steps are performed in a different order or not at all. Some steps may additionally include additional features not mentioned above.

In some examples, video conference device 115-h may transmit video indication 405 (e.g., a video feed of the patient and/or clinician), and the video conference server 135-b may receive video indication 405 of a video conference between a patient and a clinician. Video conference server 135-b may receive medical information indication 410 associated with the patient to be displayed during the video conference. Medical information indication 410 may include characteristics such as orientation of the medical information (e.g., horizontal direction or vertical direction), size of the medical information relative to a size of the video image, whether the medical information is dynamically or statically updated, or a combination thereof. These characteristics of the medical information may be determined from metadata associated with the medial information. Medical information indication 410 may include medical data associated with the patient and may be represented as a chart, a graph, a report, an image, a live data stream, or a combination thereof.

Medical information indication 410 may also be an example of a request for the medical information to be displayed that is received from at least one of the clinician, the patient, or both. In some cases, the medical information to be displayed may be based on medical information indication 410 (e.g., a request for medical information). Medical information indication 410 may also be an example of the actual medial information to be displayed that is retrieved from a database, a sensor associated with the patient, an input by the patient or the clinician during the video conference, or a combination of these sources. In other examples, medical information indication 410 may be an example of transmitting an indication of the medical information that was displayed during the video conference to a server for storage.

At block 415, video conference server 135-b may configure the viewing window (e.g., viewing screen) of the video conference device 115-h. For example, video conference server 135-b may configure a viewing window to display the video image and the medical information during the video conference. In some cases, video conference server 135-b may configure the viewing window to display the video image and the medical information in the viewing window according to the configuration. The configuration may be conveyed to the video conference device 115-h through control signaling or any other communication means.

At block 420, video conference server 135-b may arrange the viewing window. For example, video conference server 135-b may arrange a location of the medical information with respect to a location of the video image within the viewing window based on a characteristic of the medical information. In other examples, video conference server 135-b may arrange the video image in a first portion of the viewing window and arrange the medical information in a second portion of the viewing window. The location of the video image in the first portion may be different than the location of the medical information in the second portion (e.g., non-overlapping configuration). In some examples, video conference server 135-b may arrange the video image and the medical information such that the medical information at least partially overlaps the video image. Arranging the viewing window may be part of the viewing window configuration described with respect to block 415, and the arrangement may be conveyed to the video conference device 115-h in a similar manner.

At block 425, video conference server 135-b may modify the viewing window. For example, video conference server 135-b may modify a size of the video image based at least in part on a size of the medical information relative to the size of the video image, an orientation of the medical information, or both. In other examples, video conference server 135-b may adjust a resolution of the video image based at least in part on a resolution of the medical information. Modifying the viewing window may be part of the viewing window configuration described with respect to block 415, and the arrangement may be conveyed to the video conference device 115-h in a similar manner.

In some examples, video conference device 115-h may transmit annotation indication 430 (e.g., note). Video conference server 135-b may receive annotation indication 430 and may receive an indication of an annotation to the medical information from the clinician, the patient, or both. At block 435, annotation indication 430 may be arranged to display the medical information in the viewing window.

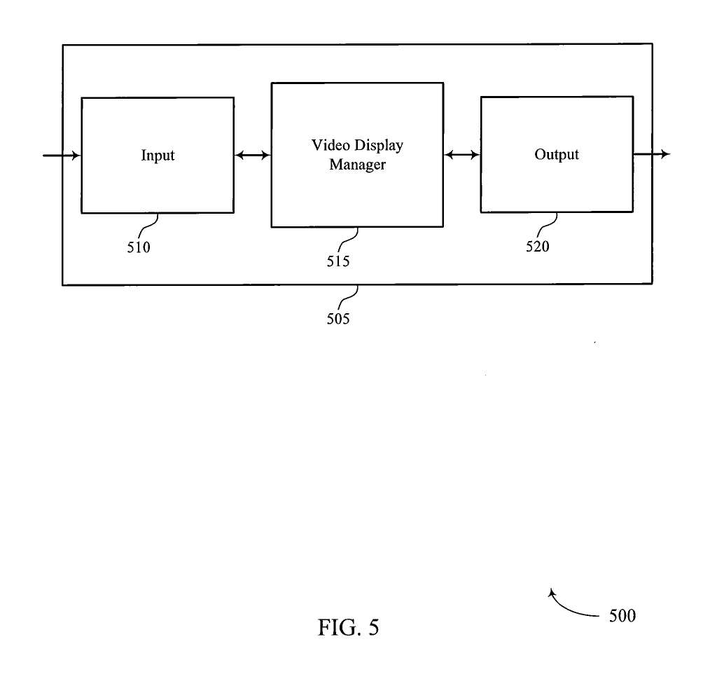

FIG. 5 shows a block diagram 500 of a device 505 that supports doctor-patient video chat storyboarding in accordance with aspects of the present disclosure. Device 505 may be an example of aspects of a video conference server as described herein. Device 505 may include input 510, video display manager 515, and output 520. Device 505 may also include a processor. Each of these components may be in communication with one another (e.g., via one or more buses).

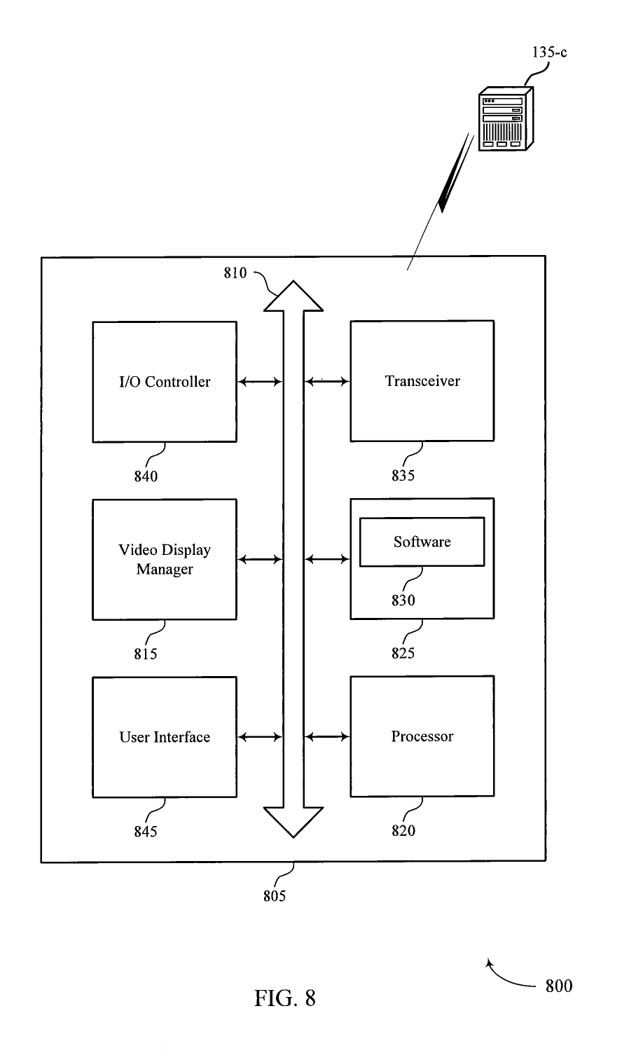

Video display manager 515 may be an example of aspects of the video display manager 815 described with reference to FIG. 8.

Video display manager 515 and/or at least some of its various sub-components may be implemented in hardware, software executed by a processor, firmware, or any combination thereof. If implemented in software executed by a processor, the functions of the video display manager 515 and/or at least some of its various sub-components may be executed by a general-purpose processor, a digital signal processor (DSP), an application-specific integrated circuit (ASIC), an field-programmable gate array (FPGA) or other programmable logic device, discrete gate or transistor logic, discrete hardware components, or any combination thereof designed to perform the functions described in the present disclosure. The video display manager 515 and/or at least some of its various sub-components may be physically located at various positions, including being distributed such that portions of functions are implemented at different physical locations by one or more physical devices. In some examples, video display manager 515 and/or at least some of its various sub-components may be a separate and distinct component in accordance with various aspects of the present disclosure. In other examples, video display manager 515 and/or at least some of its various sub-components may be combined with one or more other hardware components, including but not limited to an I/O component, a transceiver, a network server, another computing device, one or more other components described in the present disclosure, or a combination thereof in accordance with various aspects of the present disclosure.

Video display manager 515 may receive a video image of a video conference between the patient and the clinician, receive an indication of medical information associated with the patient to be displayed during the video conference, and configure a viewing window to display the video image and the medical information during the video conference.

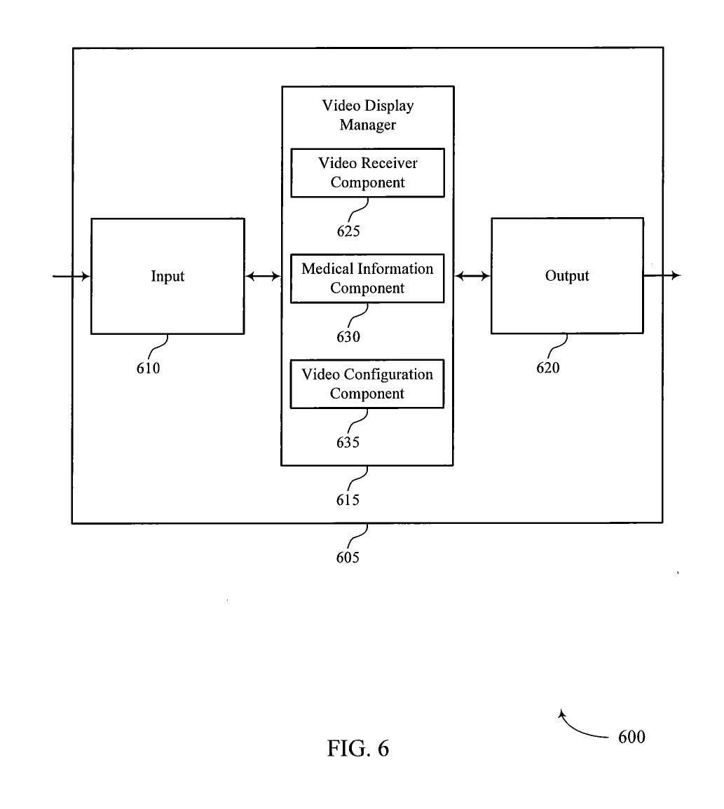

FIG. 6 shows a block diagram 600 of a device 605 that supports doctor-patient video chat storyboarding in accordance with aspects of the present disclosure. Device 605 may be an example of aspects of a device 505 or a video conference server as described with reference to FIG. 5. Device 605 may include input 610, video display manager 615, and output 620. Device 605 may also include a processor. Each of these components may be in communication with one another (e.g., via one or more buses).

Video display manager 615 may be an example of aspects of the video display manager 815 described with reference to FIG. 8.

Video display manager 615 may also include video receiver component 625, medical information component 630, and video configuration component 635.

Video receiver component 625 may receive a video image of a video conference between the patient and the clinician and receive a request for the medical information from at least one of the clinician, the patient, or both, where the medical information to be displayed is based on the received request.

Medical information component 630 may receive an indication of medical information associated with the patient to be displayed during the video conference. Medical information component 630 may also retrieve the medical information from a database, a sensor associated with the patient, an input by the patient or the clinician during the video conference, or a combination thereof. In some examples, medical information component 630 may transmit an indication of the medical information that was displayed during the video conference to a server for storage. In some cases, the characteristic of the medical information includes an orientation of the medical information, a size of the medical information relative to a size of the video image, whether the medical information is dynamically or statically updated, or a combination thereof. In some cases, the medical information includes medical data associated with the patient. In some cases, the medical information is represented as a chart, a graph, a report, an image, a live data stream, or a combination thereof.

Video configuration component 635 may configure a viewing window to display the video image and the medical information during the video conference and display the video image and the medical information in the viewing window according to the configuration. In some cases, configuring the viewing window includes modifying a size of the video image based on a size of the medical information relative to the size of the video image, an orientation of the medical information, or both. In some cases, configuring the viewing window includes adjusting a resolution of the video image based on a resolution of the medical information.

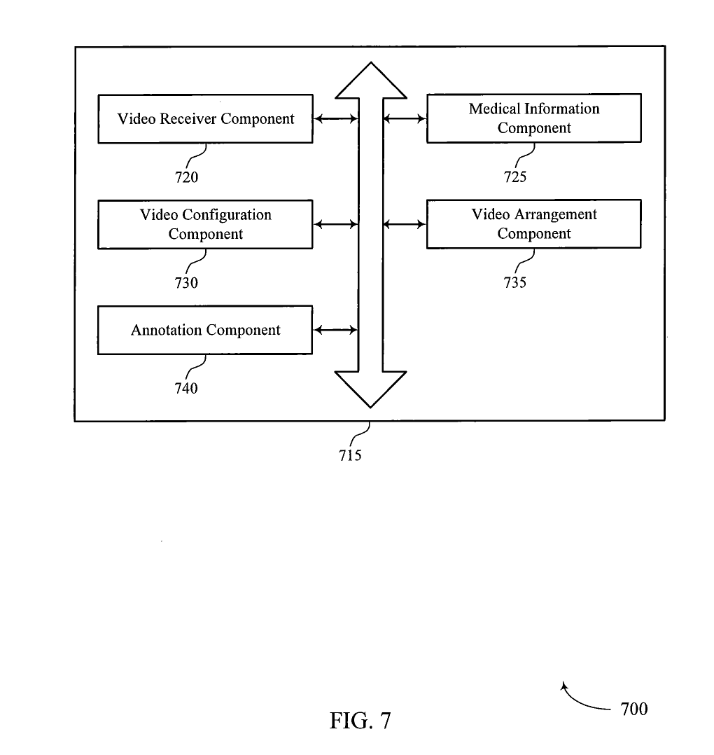

FIG. 7 shows a block diagram 700 of a video display manager 715 that supports doctor-patient video chat storyboarding in accordance with aspects of the present disclosure. The video display manager 715 may be an example of aspects of a video display manager 515, a video display manager 615, or a video display manager 815 described with reference to FIGS. 5, 6, and 8. The video display manager 715 may include video receiver component 720, medical information component 725, video configuration component 730, video arrangement component 735, and annotation component 740. Each of these modules may communicate, directly or indirectly, with one another (e.g., via one or more buses).

Video receiver component 720 may receive a video image of a video conference between the patient and the clinician and receive a request for the medical information from at least one of the clinician, the patient, or both, and where the medical information to be displayed is based on the received request.

Medical information component 725 may receive an indication of medical information associated with the patient to be displayed during the video conference. In some examples, medical information component 725 may retrieve the medical information from a database, a sensor associated with the patient, an input by the patient or the clinician during the video conference, or a combination thereof. Medical information component 725 may also transmit an indication of the medical information that was displayed during the video conference to a server for storage. In some cases, the characteristic of the medical information includes an orientation of the medical information, a size of the medical information relative to a size of the video image, whether the medical information is dynamically or statically updated, or a combination thereof. In some cases, the medical information includes medical data associated with the patient. In some cases, the medical information is represented as a chart, a graph, a report, an image, a live data stream, or a combination thereof.

Video configuration component 730 may configure a viewing window to display the video image and the medical information during the video conference and display the video image and the medical information in the viewing window according to the configuration. In some cases, configuring the viewing window includes modifying a size of the video image based on a size of the medical information relative to the size of the video image, an orientation of the medical information, or both. In some cases, configuring the viewing window includes adjusting a resolution of the video image based on a resolution of the medical information.

Video arrangement component 735 may arrange the video image in a first portion of the viewing window, arrange the medical information in a second portion of the viewing window, where the location of the video image in the first portion is different than the location of the medical information in the second portion, and arrange the video image and the medical information such that the medical information at least partially overlaps the video image. In some cases, configuring the viewing window includes arranging a location of the medical information with respect to a location of the video image within the viewing window based on a characteristic of the medical information.

Annotation component 740 may receive an indication of an annotation to the medical information from the clinician, the patient, or both and arrange the annotation to be displayed with the medical information in the viewing window.

FIG. 8 shows a diagram of a system 800 including a device 805 that supports doctor-patient video chat storyboarding in accordance with aspects of the present disclosure. Device 805 may be an example of or include the components of device 505, device 605, or a video conference server as described above, e.g., with reference to FIGS. 5 and 6. Device 805 may include components for bi-directional voice and data communications including components for transmitting and receiving communications, including video display manager 815, processor 820, memory 825, software 830, transceiver 835, I/O controller 840, and user interface 845. These components may be in electronic communication via one or more buses (e.g., bus 810).

Processor 820 may include an intelligent hardware device, (e.g., a general-purpose processor, a DSP, a central processing unit (CPU), a microcontroller, an ASIC, an FPGA, a programmable logic device, a discrete gate or transistor logic component, a discrete hardware component, or any combination thereof). The processor 820 may process information received from video conference server 135-c. In some cases, processor 820 may be configured to operate a memory array using a memory controller. In other cases, a memory controller may be integrated into processor 820. Processor 820 may be configured to execute computer-readable instructions stored in a memory to perform various functions (e.g., functions or tasks supporting doctor-patient video chat storyboarding).

Memory 825 may include random access memory (RAM) and read only memory (ROM). The memory 825 may store computer-readable, computer-executable software 830 including instructions that, when executed, cause the processor to perform various functions described herein. In some cases, the memory 825 may contain, among other things, a basic input/output system (BIOS) which may control basic hardware or software operation such as the interaction with peripheral components or devices.

Software 830 may include code to implement aspects of the present disclosure, including code to support doctor-patient video chat storyboarding. Software 830 may be stored in a non-transitory computer-readable medium such as system memory or other memory. In some cases, the software 830 may not be directly executable by the processor but may cause a computer (e.g., when compiled and executed) to perform functions described herein.

Transceiver 835 may communicate bi-directionally, via one or more antennas, wired, or wireless links as described above. For example, the transceiver 835 may represent a wireless transceiver and may communicate bi-directionally with another wireless transceiver. The transceiver 835 may also include a modem to modulate the packets and provide the modulated packets to the antennas for transmission, and to demodulate packets received from the antennas.

I/O controller 840 may manage input and output signals for device 805. I/O controller 840 may also manage peripherals not integrated into device 805. In some cases, I/O controller 840 may represent a physical connection or port to an external peripheral. In some cases, I/O controller 840 may utilize an operating system such as iOS®, ANDROID®, MS-DOS®, MS-WINDOWS®, OS/2®, UNIX®, LINUX®, or another known operating system. In other cases, I/O controller 840 may represent or interact with a modem, a keyboard, a mouse, a touchscreen, or a similar device. In some cases, I/O controller 840 may be implemented as part of a processor. In some cases, a user may interact with device 805 via I/O controller 840 or via hardware components controlled by I/O controller 840.

User interface 845 may enable a user to interact with device 805. In some embodiments, the user interface module 845 may include an audio device, such as an external speaker system, an external display device such as a display screen, or an input device (e.g., remote control device interfaced with the user interface module 845 directly or through the I/O controller module).

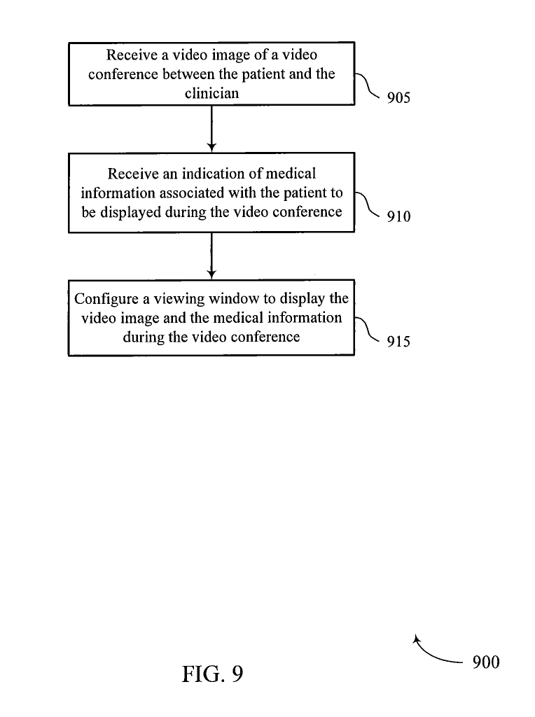

FIG. 9 shows a flowchart illustrating a method 900 for doctor-patient video chat storyboarding in accordance with aspects of the present disclosure. The operations of method 900 may be implemented by a video conference server or its components as described herein. For example, the operations of method 900 may be performed by a video display manager as described with reference to FIGS. 5 through 8. In some examples, a video conference server may execute a set of codes to control the functional elements of the device to perform the functions described below. Additionally or alternatively, the video conference server may perform aspects of the functions described below using special-purpose hardware.

At 905 the video conference server may receive a video image of a video conference between the patient and the clinician. The operations of 905 may be performed according to the methods described herein. In certain examples, aspects of the operations of 905 may be performed by a video receiver component as described with reference to FIGS. 5 through 8.

At 910 the video conference server may receive an indication of medical information associated with the patient to be displayed during the video conference. The operations of 910 may be performed according to the methods described herein. In certain examples, aspects of the operations of 910 may be performed by a medical information component as described with reference to FIGS. 5 through 8.

At 915 the video conference server may configure a viewing window to display the video image and the medical information during the video conference. The operations of 915 may be performed according to the methods described herein. In certain examples, aspects of the operations of 915 may be performed by a video configuration component as described with reference to FIGS. 5 through 8.

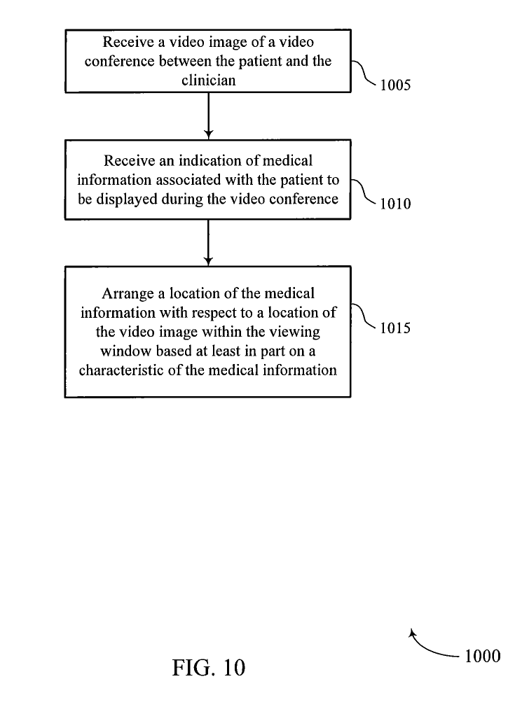

FIG. 10 shows a flowchart illustrating a method 1000 for doctor-patient video chat storyboarding in accordance with aspects of the present disclosure. The operations of method 1000 may be implemented by a video conference server or its components as described herein. For example, the operations of method 1000 may be performed by a video display manager as described with reference to FIGS. 5 through 8. In some examples, a video conference server may execute a set of codes to control the functional elements of the device to perform the functions described below. Additionally or alternatively, the video conference server may perform aspects of the functions described below using special-purpose hardware.

At 1005 the video conference server may receive a video image of a video conference between the patient and the clinician. The operations of 1005 may be performed according to the methods described herein. In certain examples, aspects of the operations of 1005 may be performed by a video receiver component as described with reference to FIGS. 5 through 8.

At 1010 the video conference server may receive an indication of medical information associated with the patient to be displayed during the video conference. The operations of 1010 may be performed according to the methods described herein. In certain examples, aspects of the operations of 1010 may be performed by a medical information component as described with reference to FIGS. 5 through 8.

At 1015 the video conference server may arrange a location of the medical information with respect to a location of the video image within the viewing window based at least in part on a characteristic of the medical information. The operations of 1020 may be performed according to the methods described herein. In certain examples, aspects of the operations of 1020 may be performed by a video arrangement component as described with reference to FIGS. 5 through 8.

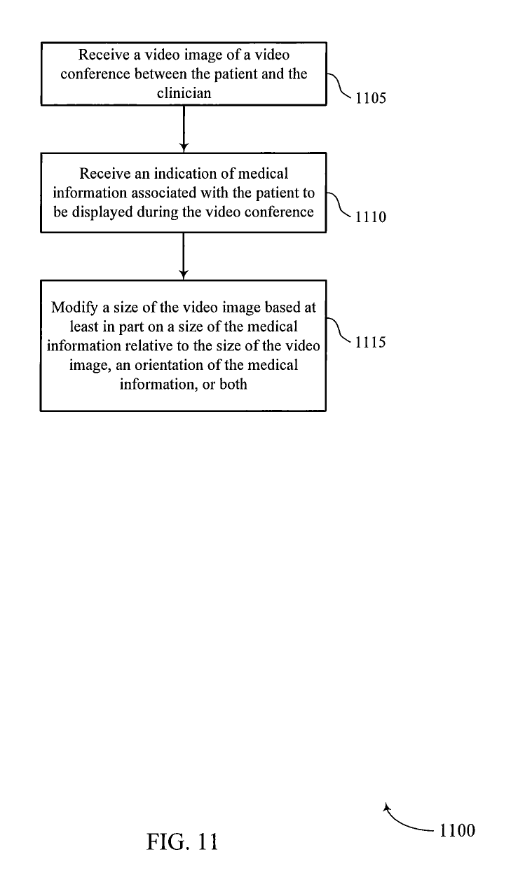

FIG. 11 shows a flowchart illustrating a method 1100 for doctor-patient video chat storyboarding in accordance with aspects of the present disclosure. The operations of method 1100 may be implemented by a video conference server or its components as described herein. For example, the operations of method 1100 may be performed by a video display manager as described with reference to FIGS. 5 through 8. In some examples, a video conference server may execute a set of codes to control the functional elements of the device to perform the functions described below. Additionally or alternatively, the video conference server may perform aspects of the functions described below using special-purpose hardware.

At 1105 the video conference server may receive a video image of a video conference between the patient and the clinician. The operations of 1105 may be performed according to the methods described herein. In certain examples, aspects of the operations of 1105 may be performed by a video receiver component as described with reference to FIGS. 5 through 8.

At 1110 the video conference server may receive an indication of medical information associated with the patient to be displayed during the video conference. The operations of 1110 may be performed according to the methods described herein. In certain examples, aspects of the operations of 1110 may be performed by a medical information component as described with reference to FIGS. 5 through 8.

At 1115 the video conference server may modify a size of the video image based at least in part on a size of the medical information relative to the size of the video image, an orientation of the medical information, or both. The operations of 1120 may be performed according to the methods described herein. In certain examples, aspects of the operations of 1120 may be performed by a video configuration component as described with reference to FIGS. 5 through 8.

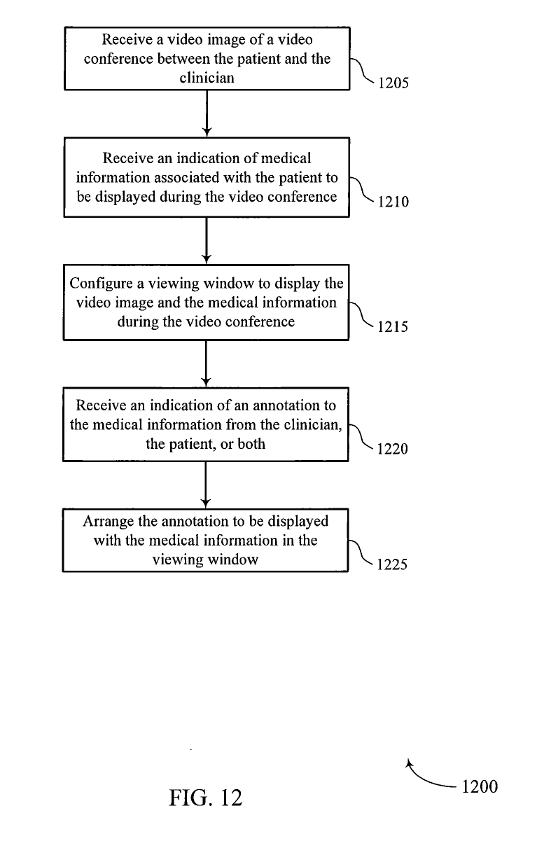

FIG. 12 shows a flowchart illustrating a method 1200 for doctor-patient video chat storyboarding in accordance with aspects of the present disclosure. The operations of method 1200 may be implemented by a video conference server or its components as described herein. For example, the operations of method 1200 may be performed by a video display manager as described with reference to FIGS. 5 through 8. In some examples, a video conference server may execute a set of codes to control the functional elements of the device to perform the functions described below. Additionally or alternatively, the video conference server may perform aspects of the functions described below using special-purpose hardware.

At 1205 the video conference server may receive a video image of a video conference between the patient and the clinician. The operations of 1205 may be performed according to the methods described herein. In certain examples, aspects of the operations of 1205 may be performed by a video receiver component as described with reference to FIGS. 5 through 8.

At 1210 the video conference server may receive an indication of medical information associated with the patient to be displayed during the video conference. The operations of 1210 may be performed according to the methods described herein. In certain examples, aspects of the operations of 1210 may be performed by a medical information component as described with reference to FIGS. 5 through 8.

At 1215 the video conference server may configure a viewing window to display the video image and the medical information during the video conference. The operations of 1215 may be performed according to the methods described herein. In certain examples, aspects of the operations of 1215 may be performed by a video configuration component as described with reference to FIGS. 5 through 8.

At 1220 the video conference server may receive an indication of an annotation to the medical information from the clinician, the patient, or both. The operations of 1220 may be performed according to the methods described herein. In certain examples, aspects of the operations of 1220 may be performed by an annotation component as described with reference to FIGS. 5 through 8.

At 1225 the video conference server may arrange the annotation to be displayed with the medical information in the viewing window. The operations of 1225 may be performed according to the methods described herein. In certain examples, aspects of the operations of 1225 may be performed by an annotation component as described with reference to FIGS. 5 through 8.

It should be noted that the methods described above describe possible implementations, and that the operations and the steps may be rearranged or otherwise modified and that other implementations are possible. Furthermore, aspects from two or more of the methods may be combined.

The description set forth herein, in connection with the appended drawings, describes example configurations and does not represent all the examples that may be implemented or that are within the scope of the claims. The term “exemplary” used herein means “serving as an example, instance, or illustration,” and not “preferred” or “advantageous over other examples.” The detailed description includes specific details for the purpose of providing an understanding of the described techniques. These techniques, however, may be practiced without these specific details. In some instances, well-known structures and devices are shown in block diagram form in order to avoid obscuring the concepts of the described examples.

In the appended figures, similar components or features may have the same reference label. Further, various components of the same type may be distinguished by following the reference label by a dash and a second label that distinguishes among the similar components. If just the first reference label is used in the specification, the description is applicable to any one of the similar components having the same first reference label irrespective of the second reference label.

Information and signals described herein may be represented using any of a variety of different technologies and techniques. For example, data, instructions, commands, information, signals, bits, symbols, and chips that may be referenced throughout the above description may be represented by voltages, currents, electromagnetic waves, magnetic fields or particles, optical fields or particles, or any combination thereof.

The various illustrative blocks and modules described in connection with the disclosure herein may be implemented or performed with a general-purpose processor, a digital signal processor (DSP), an ASIC, an field programmable gate array (FPGA) or other programmable logic device, discrete gate or transistor logic, discrete hardware components, or any combination thereof designed to perform the functions described herein. A general-purpose processor may be a microprocessor, but in the alternative, the processor may be any conventional processor, controller, microcontroller, or state machine. A processor may also be implemented as a combination of computing devices (e.g., a combination of a DSP and a microprocessor, multiple microprocessors, one or more microprocessors in conjunction with a DSP core, or any other such configuration). A processor may in some cases be in electronic communication with a memory, where the memory stores instructions that are executable by the processor. Thus, the functions described herein may be performed by one or more other processing units (or cores), on at least one integrated circuit (IC). In various examples, different types of ICs may be used (e.g., Structured/Platform ASICs, an FPGA, or another semi-custom IC), which may be programmed in any manner known in the art. The functions of each unit may also be implemented, in whole or in part, with instructions embodied in a memory, formatted to be executed by one or more general or application-specific processors.

The functions described herein may be implemented in hardware, software executed by a processor, firmware, or any combination thereof. If implemented in software executed by a processor, the functions may be stored on or transmitted over as one or more instructions or code on a computer-readable medium. Other examples and implementations are within the scope of the disclosure and appended claims. For example, due to the nature of software, functions described above may be implemented using software executed by a processor, hardware, firmware, hardwiring, or combinations of any of these. Features implementing functions may also be physically located at various positions, including being distributed such that portions of functions are implemented at different physical locations. Also, as used herein, including in the claims, “or” as used in a list of items (for example, a list of items prefaced by a phrase such as “at least one of” or “one or more of”) indicates an inclusive list such that, for example, a list of at least one of A, B, or C means A or B or C or AB or AC or BC or ABC (i.e., A and B and C). Also, as used herein, the phrase “based on” shall not be construed as a reference to a closed set of conditions. For example, an exemplary step that is described as “based on condition A” may be based on both a condition A and a condition B without departing from the scope of the present disclosure. In other words, as used herein, the phrase “based on” shall be construed in the same manner as the phrase “based at least in part on.”

Computer-readable media includes both non-transitory computer storage media and communication media including any medium that facilitates transfer of a computer program from one place to another. A non-transitory storage medium may be any available medium that can be accessed by a general purpose or special purpose computer. By way of example, and not limitation, non-transitory computer-readable media may comprise RAM, ROM, electrically erasable programmable read only memory (EEPROM), compact disk (CD) ROM or other optical disk storage, magnetic disk storage or other magnetic storage devices, or any other non-transitory medium that may be used to carry or store desired program code means in the form of instructions or data structures and that may be accessed by a general-purpose or special-purpose computer, or a general-purpose or special-purpose processor. Also, any connection is properly termed a computer-readable medium. For example, if the software is transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, digital subscriber line (DSL), or wireless technologies such as infrared, radio, and microwave are included in the definition of medium. Disk and disc, as used herein, include CD, laser disc, optical disc, digital versatile disc (DVD), floppy disk and Blu-ray disc where disks usually reproduce data magnetically, while discs reproduce data optically with lasers. Combinations of the above are also included within the scope of computer-readable media.

The description herein is provided to enable a person skilled in the art to make or use the disclosure. Various modifications to the disclosure will be readily apparent to those skilled in the art, and the generic principles defined herein may be applied to other variations without departing from the scope of the disclosure. Thus, the disclosure is not limited to the examples and designs described herein, but is to be accorded the broadest scope consistent with the principles and novel features disclosed herein.

1. A method for video conferencing between a patient and a clinician, comprising:
receiving a video image of a video conference between the patient and the clinician;
receiving an indication of medical information associated with the patient to be displayed during the video conference; and
configuring a viewing window to display the video image and the medical information during the video conference based at least in part on a characteristic of the medical information, wherein the characteristic of the medical information comprises a display orientation of the medical information with respect to the video image in the viewing window.

2. The method of claim 1, wherein configuring the viewing window comprises:
arranging a location of the medical information with respect to a location of the video image within the viewing window based at least in part on the characteristic of the medical information.

3. The method of claim 2, wherein the characteristic of the medical information further comprises a size of the medical information relative to a size of the video image, whether the medical information is dynamically or statically updated, or a combination thereof.

4. The method of claim 2, further comprising:
arranging the video image in a first portion of the viewing window; and
arranging the medical information in a second portion of the viewing window, wherein the location of the video image in the first portion is different than the location of the medical information in the second portion.

5. The method of claim 2, further comprising:
arranging the video image and the medical information such that the medical information at least partially overlaps the video image.

6. The method of claim 1, wherein configuring the viewing window comprises:
modifying a size of the video image based at least in part on a size of the medical information relative to the size of the video image, the orientation of the medical information, or both.

7. The method of claim 1, wherein configuring the viewing window comprises:
adjusting a resolution of the video image based at least in part on a resolution of the medical information.

8. The method of claim 1, further comprising:
receiving a request for the medical information from at least one of the clinician, the patient, or both, and wherein the medical information to be displayed is based at least in part on the received request.

9. The method of claim 1, further comprising:
receiving an indication of an annotation to the medical information from the clinician, the patient, or both; and
arranging the annotation to be displayed with the medical information in the viewing window.

10. The method of claim 1, further comprising:
retrieving the medical information from a database, a sensor associated with the patient, an input by the patient or the clinician during the video conference, or a combination thereof.

11. The method of claim 1, further comprising:
transmitting an indication of the medical information that was displayed during the video conference to a server for storage.

12. The method of claim 1, further comprising:
displaying the video image and the medical information in the viewing window according to the configuration.

13. The method of claim 1, wherein the medical information comprises medical data associated with the patient.

14. The method of claim 1, wherein the medical information is represented as a chart, a graph, a report, an image, a live data stream, or a combination thereof.

15. An apparatus for video conferencing between a patient and a clinician, comprising:
a processor;
memory in electronic communication with the processor; and
instructions stored in the memory and executable by the processor to cause the apparatus to:
receive a video image of a video conference between the patient and the clinician;
receive an indication of medical information associated with the patient to be displayed during the video conference; and
configure a viewing window to display the video image and the medical information during the video conference based at least in part on a characteristic of the medical information, wherein the characteristic of the medical information comprises a display orientation of the medical information with respect to the video image in the viewing window.

16. The apparatus of claim 15, wherein the instructions to configure the viewing window are executable by the processor to cause the apparatus to:
arrange a location of the medical information with respect to a location of the video image within the viewing window based at least in part on the characteristic of the medical information, wherein the characteristic of the medical information further comprises a size of the medical information relative to a size of the video image, whether the medical information is dynamically or statically updated, or a combination thereof.

17. The apparatus of claim 16, wherein the instructions are further executable by the processor to cause the apparatus to:
arrange the video image in a first portion of the viewing window; and
arrange the medical information in a second portion of the viewing window, wherein the location of the video image in the first portion is different than the location of the medical information in the second portion.

18. The apparatus of claim 16, wherein the instructions are further executable by the processor to cause the apparatus to:
arrange the video image and the medical information such that the medical information at least partially overlaps the video image.

19. The apparatus of claim 15, wherein the instructions are further executable by the processor to cause the apparatus to:
receive an indication of an annotation to the medical information from the clinician, the patient, or both; and
arrange the annotation to be displayed with the medical information in the viewing window.

20. A non-transitory computer readable medium storing code for video conferencing between a patient and a clinician, the code comprising instructions executable by a processor to:
receive a video image of a video conference between the patient and the clinician;
receive an indication of medical information associated with the patient to be displayed during the video conference; and
configure a viewing window to display the video image and the medical information during the video conference based at least in part on a characteristic of the medical information, wherein the characteristic of the medical information comprises a display orientation of the medical information with respect to the video image in the viewing window.
